(19) United States
(12) Patent Application Publication
(10) Pub. No.: US 2002/0082622 A1
(43) Pub. Date: Jun. 27, 2002

(54) Collapsed deployable soft tissue anchor for repairing soft tissue to bone

(76) Inventor: David Lee Kane, League City, TX (US)

Correspondence Address: Vanessa B. Pierce, Parsons Behle & Latimer, One Utah Center, 201 South Main Street, Suite 1800, Salt Lake City, UT, 84145-0898, US

(21) Appl. No.: 10/010,017

(22) Filed: Dec. 5, 2001

Related U.S. Application Data
(60) Provisional application No. 60/252,955, filed on Nov. 24, 2000.

(51) Int. Cl. A61B 17/08

(52) U.S. Cl. 606/151; 606/213

(57) ABSTRACT

A soft tissue anchor used to repair or reattach soft tissue to a bone surface. The device includes one or a series of tongs of various lengths and arrangements attached to an anchor portion that extends into a bone tunnel. The tongs are compressed within a compression sleeve and deployed, thereby securing soft tissue to bone, when the compression sleeve is moved proximally along a pushing rod.

### I. BACKGROUND

1. Field of Invention

This invention relates to the repair or reattachment of soft tissue to a bone surface as is commonly required within the practice of orthopedic surgery or other like practice.

2. Description of Related Art

Orthopedic surgeons are often challenged with repairing or reattaching soft tissue that has been torn or severed from a bone surface. In general, such repair requires that a bone tunnel be drilled into the bone underlying the soft tissue that is to be repaired or attached to the bone. Typically, once a tunnel has been drilled into the bone, the distal end of a tissue-securing device is press-fit into the bone tunnel, while the proximal end of the tissue-securing device secures the tissue to the surface of the bone. Such tissue-securing devices conventionally employ either tack-like structures that secure the tissue to the bone, or anchors that require sutures, which must be tied to the soft tissue. Both of these tissue-securing modalities suffer from inherent problems.

First, the tack-like structures require a tissue securing head that must be broad enough to provide adequate purchase of the soft tissue that is being secured to the bone surface. Such a broad tack head makes the use of the device difficult within the confines of a small joint space. In addition, it is difficult to employ the tack through the small diameter cannulae typically used to provide access into an anatomic joint space or cavity. Indeed, there are times when the size of the tack head alone makes its use prohibitively difficult. Further, the tack cannot always be introduced at an angle perpendicular to the bone surface and as such, the tack does not sit flatly upon the tissue that is to be anchored to the bone.

Second, tissue securing devices requiring sutures employ a trailing suture that must be passed through the tissue once the anchor has been impacted or screwed into the bone. In the case of an arthroscopic procedure, for example, which is performed entirely through cannulae, this passing of the suture through the tissue presents a difficult and time-consuming portion of the procedure and requires that additional portals be established into the joint. These portals are used for passing instruments into the joint or anatomical space, which in turn are used to pass the sutures through the tissue to be repaired. Once through the tissue, the sutures must then be tied into knots to secure the tissue to bone. The knot tying procedure is both difficult and time consuming, and studies show that such knots are prone to failure.

### II. SUMMARY OF THE INVENTION

In accordance with the present disclosure, the soft tissue anchor device comprises a tong portion that contains at least one collapsible tissue anchoring tong and an anchor portion secured into a previously prepared bone tunnel. The soft tissue anchor device preferably includes two tissue anchoring tongs that deploy into a tissue holding state through withdrawal of a protective compressive sleeve that otherwise maintains the tongs in an undeployed state. For example, once the soft tissue anchor device is placed within the joint or anatomical space, the tissue anchoring tongs are deployed through retraction, such as through a sliding or unscrewing mechanism, of the protective compressive sleeve in a proximal direction. The tissue anchoring tongs deploy upon proximal retraction of the compressive sleeve, and the bone anchor portion of the soft tissue anchor device is then driven into a previously prepared bone tunnel by way of a central pushing rod. The tongs are then free to gain a secure purchase of the soft tissue that is to be secured to the bone surface.

It will be appreciated that, by inserting the compressed soft tissue anchor device into the joint or anatomical cavity, prior to deployment of the tongs, the tissue anchor device can be employed through a narrow cannula and thus, can be used within a very small area. Since the soft tissue anchor device is compressed until it is within the joint, the anchor is delivered in a smaller state than when it is deployed. Thus, the soft tissue anchor device can be used within areas where space and visualization are at a minimum and in areas where much larger tissue securing devices, or their accompanying cannulae, cannot be used. In fact, by using this design, the diameter of the cannula used to access the joint may be reduced by as much as 3 to 4 mm, which in many areas of the body is essential for adequate access and visualization.

Further, since no sutures are employed, the procedure can be accomplished through a minimum of established portals and thus requires neither suture passage through the tissue to be repaired, nor the need for knot-tying instruments and know-how.

Finally, by offering tissue-anchoring tongs of varying length from one side of the anchor to the other, the soft tissue anchor device can be driven into bone at an angle other than perpendicular. In fact, the, resulting in fixation can even be parallel to the bone surface. For example, in one embodiment, a tong on one side of the anchor device is longer than on an opposite side, thereby facilitating angled entry into a bone tunnel.

Preferably, the tissue anchor device is employed via traditional cannulation techniques, including using a cannulated drill to prepare the bone tunnel, and then seating the tissue anchor device over a guide wire or pin. Once the anchor is seated, the guide wire or pin is withdrawn and the procedure is completed.

It will be appreciated that alternate embodiments of a tissue anchor device in accordance with the present disclosure may employ single or multiple tissue anchoring tongs arranged in various patterns and manufactured from materials including, but not limited to, bioabsorbable polymers or plastics.

To secure an anatomical soft tissue device to a bone surface, particularly within a joint space, a cannula or access portal is placed from outside the joint to within the joint while visualizing the interior of the joint by way of an arthroscopic camera placed within a second and separate portal or cannula. Through a repair portal or cannula, a guide wire is placed and used to purchase (grip) the tissue to be repaired. In particular, the guide wire is used to pierce the tissue and then place it on the bone surface by placing the tip of the wire on the bone surface in the area that is to receive the soft tissue anchor. A cannulated drill is then placed over the guide wire and secured to the wire and together the guide wire and the drill are drilled into the bone through the soft tissue. The guide wire is left in place and the cannulated drill is withdrawn. At this point, a cannulated, pre-loaded (compressed) soft tissue anchor device in accordance with the present disclosure and its cannulated deployment instrumentation, comprising a pushing rod and a compression sleeve, are inserted over the guide wire and pushed to the level of the soft tissue to be repaired.

Once within the joint, the compression sleeve is withdrawn (retracted proximally), through either a pull-out or screw-out mechanism that will then allow the tissue anchoring tongs to deploy. At this point the anchor is pushed by way of a mallet blow to the proximal end of the pushing rod, into the bone hole or tunnel, and seated. The deployed tongs will have purchased the tissue and will hold it securely. The pushing rod is then unscrewed or dislodged from the anchor and the guide wire and pushing rod are withdrawn from the joint.

### III. BRIEF DESCRIPTION OF THE DRAWINGS

In order to more fully understand the manner in which the above-recited and other advantages of a soft tissue anchor device in accordance with the present disclosure are obtained, a more particular description will be rendered by reference to specific embodiments that are illustrated in the appended drawings. Understanding that these drawings depict only typical embodiments and are not therefore to be considered to be limiting, the soft tissue anchor device in its presently understood best mode for making and using the same will be described and explained with additional specificity and detail through the use of the accompanying drawings in which:

FIG. 1 depicts a cross-sectional side view of an embodiment of a soft tissue anchor device in accordance with the present disclosure;

FIG. 2 depicts a cross-sectional side view of an embodiment of a pushing rod in accordance with the present disclosure;

FIG. 3 depicts a cross-sectional side view of an embodiment of an outer compression sleeve in accordance with the present disclosure;

FIG. 4 depicts a cross-sectional side view of the tissue anchor, pushing rod and compression sleeve from FIGS. 1-3, above, as assembled and un-deployed;

FIG. 5 depicts a cross-sectional side view of the tissue anchor device, pushing rod and compression sleeve, from FIG. 4 in a deployed state;

FIG. 6 depicts a cross-sectional side view of an alternate embodiment of an undeployed tissue anchor device, pushing rod, and compression sleeve;

FIG. 7 depicts a cross-sectional side view of an alternate embodiment of a deployed tissue anchor, pushing rod and compression sleeve;

FIG. 8 depicts a three-dimensional view of the un-deployed tissue anchor device, pushing rod and compression sleeve from FIG. 6, above, and including a proximal migration block;

FIG. 9 depicts a side view deployed tissue anchor device; and

FIG. 10 depicts an embodiment of a deployed tissue anchor device inserted at an angle.

### IV. DETAILED DESCRIPTION OF THE DRAWINGS

In a preferred embodiment, a soft tissue anchor device includes at least one deployable tong and a tissue anchor portion. For example, FIG. 1 illustrates a soft tissue anchor device (10), which includes two deployable tongs (12) attached to a tissue anchor portion (14). The deployable tongs are preferably wing or crescent-shaped such that they attain secure purchase with the soft tissue. One of skill in the art will appreciate that deployable tongs of alternate shapes that secure soft tissue to the bone will fall within the scope of the present disclosure. Further, as described in more detail below with respect to FIG. 10, in an alternate embodiment, the deployable tongs include an asymmetrical arrangement.

The terms “proximal” and “proximally” will be used herein to describe that portion of a device that is closer to skin's surface, or more superficial in the patient, while the terms “distal” and “distally” will be used to describe that portion of a device that is closer to the bone, or deeper in the patient. One of skill in the art will appreciate that these terms may be used differently in reference to other fields or medical devices.

As illustrated in FIG. 1, the deployable tongs are situated proximally in the soft tissue anchor device (10), and the anchor portion (14) is situated distally. Toward the distal end of the anchor portion (14), lies a ribbed portion (16). In use, the ribbed portion seats within a bone tunnel. Preferably, the ribs of the ribbed portion are set in a first and second series. The first series has retrograde ribs (18) that once seated within a bone tunnel, will not easily travel in a retrograde (reverse) direction. The second series has longitudinal ribs (20) that prevent rotational movement of the soft tissue anchor device once seated within the bone tunnel.

Proximal to the ribbed portion (16) lies a blocking ridge (28). The blocking ridge preferably has a diameter that exceeds the diameter of the ribbed portion and the bone tunnel thereby preventing the soft tissue anchor device from traveling too deep into bone tunnel and potentially shearing off the tissue anchoring tongs.

Distal to the ribbed portion (16), the soft tissue anchor includes rounded portion (26). The rounded portion facilitates passage of the soft tissue anchor device through the soft tissue to be repaired or affixed to a bone surface.

The soft tissue anchor device includes a cannulation channel (24), which permits the passage of a guide wire through the soft tissue anchor device. The cannulation channel also directs a bone drill to the appropriate area on the bone to be drilled and subsequently to be filled with the soft tissue anchor.

At the most proximal portion of the soft tissue anchor device, there is a male threaded mounting tab (22). The threaded mounting tab is preferably secured to a pushing rod (30) via a female threaded portion (32) as illustrated in FIG. 2. One of skill in the art will appreciate that the soft tissue anchor device can be secured to the pushing rod via alternate securing mechanisms such as, but not limited to, a hex head or morris taper.

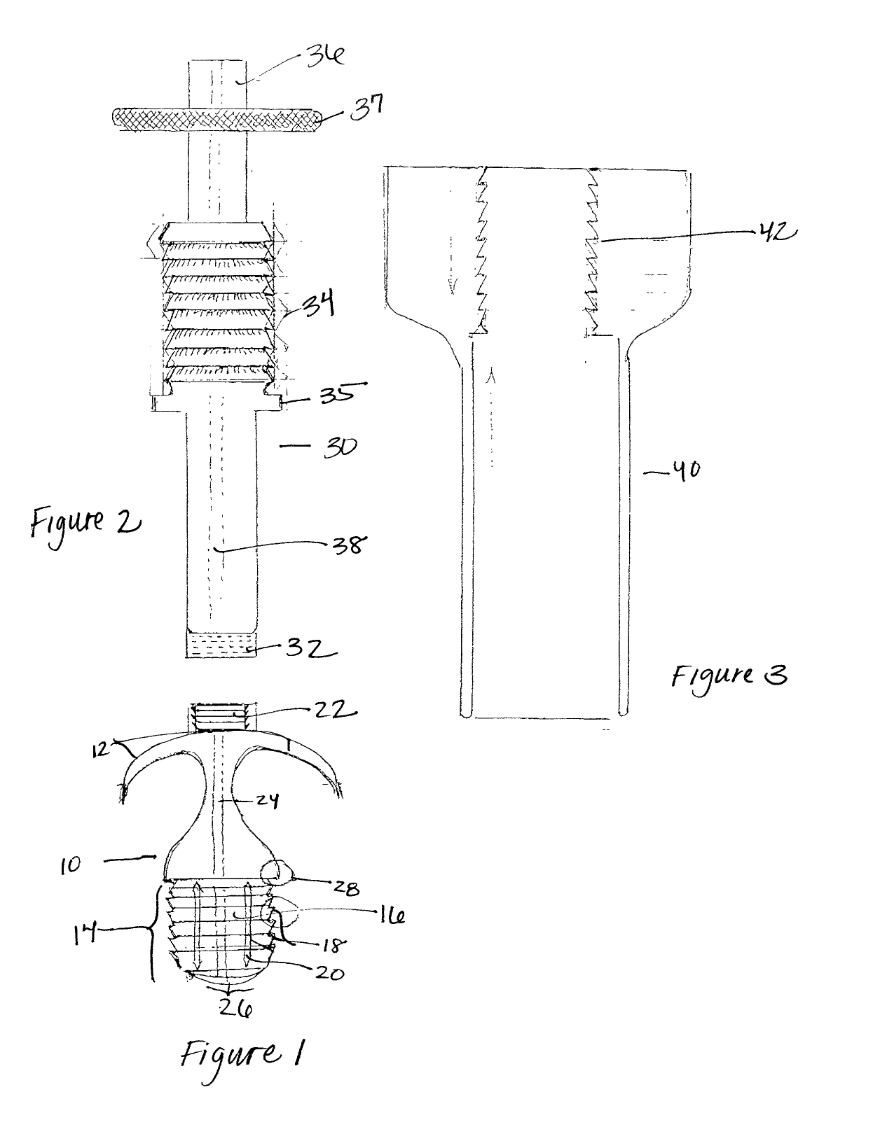

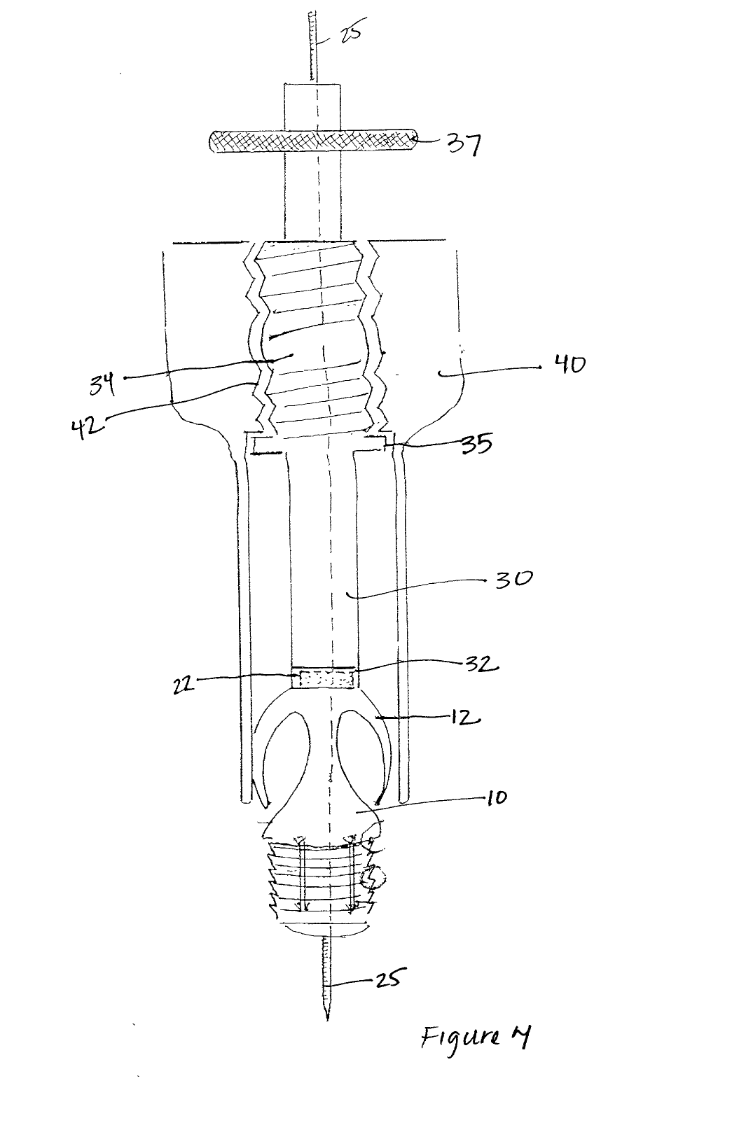

Proximally on the pushing rod (30) illustrated in FIG. 2, lies a threaded area (34) that mounts the pushing rod to an outer compression sleeve (40), illustrated in FIG. 3. Distal to the threaded area (34) lies a distal blocking sleeve (35) that prevents the outer compression sleeve (40), illustrated in FIG. 3, from extending distally off of the pushing rod (30). Proximal to the threaded area (34), lies a handle (36) and a proximal blocking sleeve (37) that prevents the compression sleeve (30) of FIG. 3 from coming off of the pushing rod (30) in a proximal direction. The pushing rod (30) also includes a cannulation channel (38). The pushing rod cannulation channel communicates with the soft tissue anchor cannulation channel to allow passage of a guide wire over which the soft tissue anchor device and pushing rod travel.

FIG. 3 illustrates the compression sleeve (40), which, when placed over the tissue anchoring tongs (12) illustrated in FIG. 1, compresses the tongs into an undeployed configuration. (See FIGS. 4 and 6, for example.) The proximal portion of the compression sleeve includes threaded area (42), which secures the compression sleeve in proper orientation to the pushing rod (30) illustrated in FIG. 2.

In FIG. 4, an embodiment of a soft tissue anchor device (10), a pushing rod (30), and a compression sleeve (40) is illustrated in an assembled fashion with the tissue anchoring tongs (12) compressed, and thus un-deployed, within the compression sleeve (40). The soft tissue anchor device (10) is affixed through male threaded mounting tab (22) to the pushing rod (30) via the female threaded portion (32) at the distal end of the pushing rod.

The pushing rod (30) is affixed to the compression sleeve (40) by way of the threaded areas (34) and (42) respectively. The proximal and distal blocking sleeves (37) and (35), respectively, keep the pushing rod (30) and compression sleeve (40) from becoming disengaged. Also shown is the guide wire (25), which proceeds through the cannulation channels of the pushing rod (30) and the soft tissue anchor device (10).

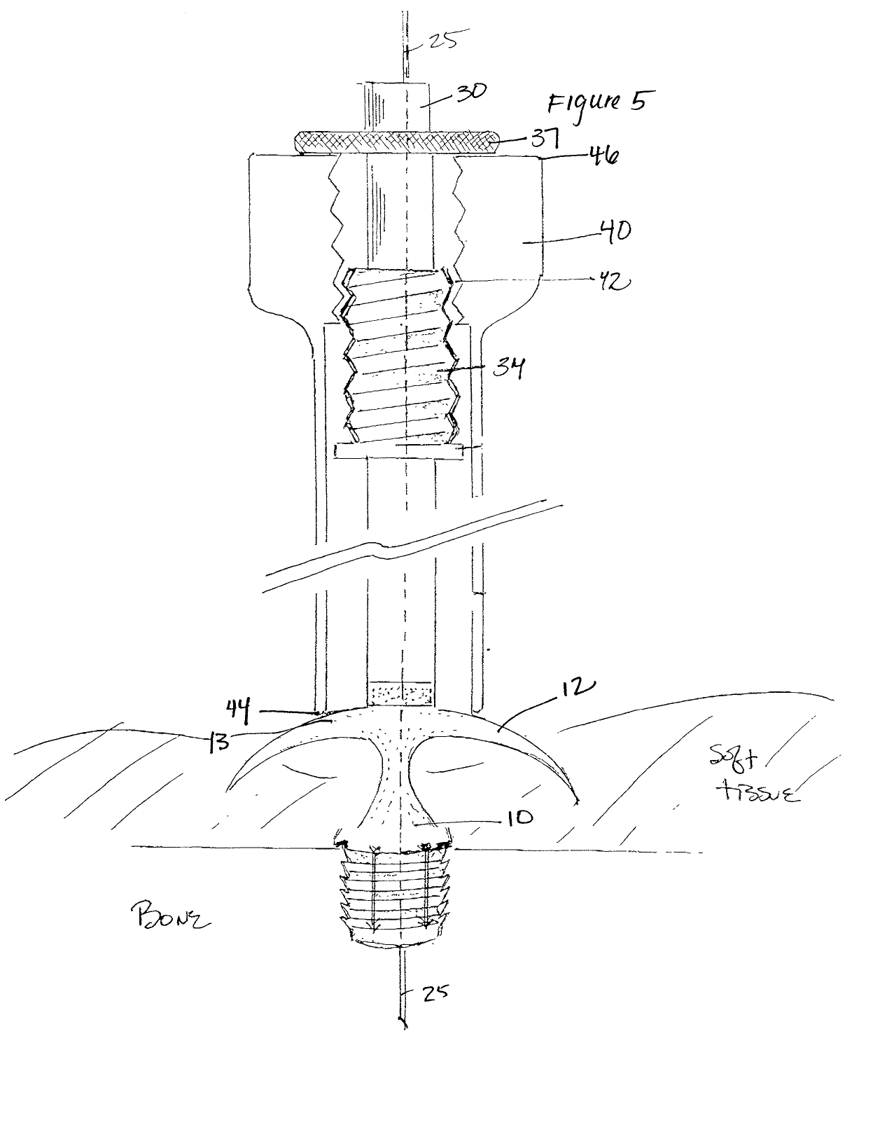

FIG. 5 illustrates an embodiment of the soft tissue anchor device, pushing rod, and compression sleeve, described in FIG. 4, wherein the tissue anchoring tongs (12) have been deployed after proximal retraction of the compression sleeve (40). The most distal portion (44) of the compression sleeve (40) appears to be resting on the most proximal portion (13) of the tissue anchoring tongs (12).

The most proximal portion (46) of the compression sleeve (40) is in direct contact with the proximal blocking sleeve (37) of the pushing rod (30), thus preventing the compression sleeve (40) from becoming disengaged from the pushing rod (30). The guide wire (25) is shown engaged through the pushing rod (30) and the tissue anchor device (10). The compression sleeve (40) has been withdrawn proximally from the pushing rod (30) through unscrewing or turning threaded areas (34) and (42).

FIG. 6 illustrates an embodiment of a soft tissue anchor device (110) employing an alternate embodiment of a compression sleeve and pushing rod. The compression sleeve (140) moves proximally and distally over the pushing rod (130) through the use of a “line-to-line” sliding mechanism (150). The anchoring tongs (112) are depicted un-deployed from the compressing force of the compression sleeve (140). To prevent inadvertent deployment of the anchoring tongs (112) through proximal migration of the compression sleeve (140), a proximal migration block (152) may be placed upon the pushing rod handle (136). The distal blocking sleeve (135) on the pushing rod (130) prevents the compression sleeve (140) from migrating too far distally off the end of the pushing rod (130).

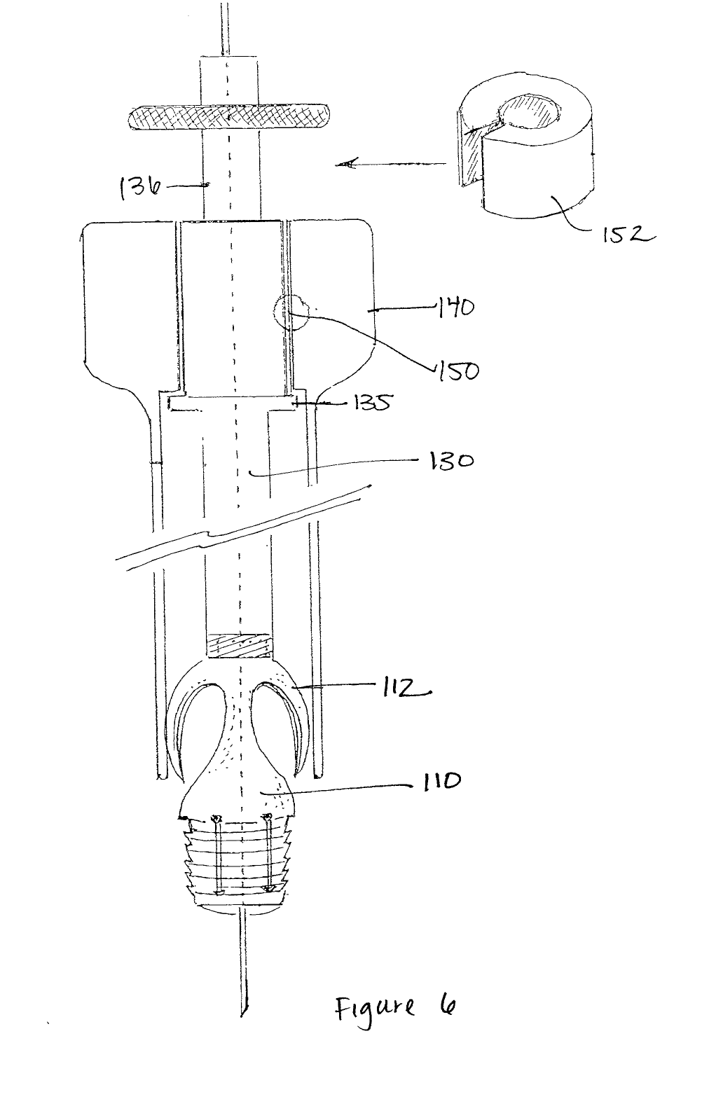

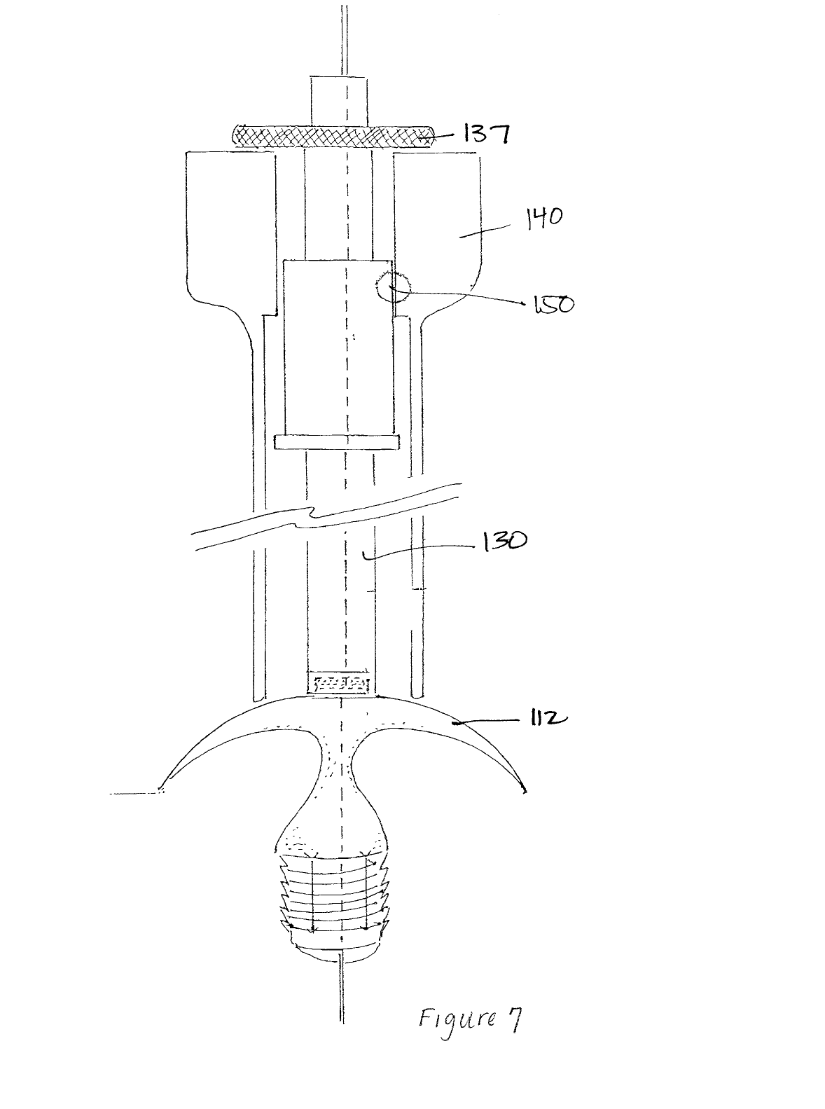

FIG. 7 illustrates the tissue anchoring tongs (112) from FIG. 6 deployed through proximal migration of the compression sleeve (140) relative to the pushing rod (130) via sliding mechanism (150). Proximal migration block (not illustrated) has been removed to allow such migration. The proximal blocking sleeve (137) prevents the compression sleeve (140) becoming disengaged from the pushing rod (130).

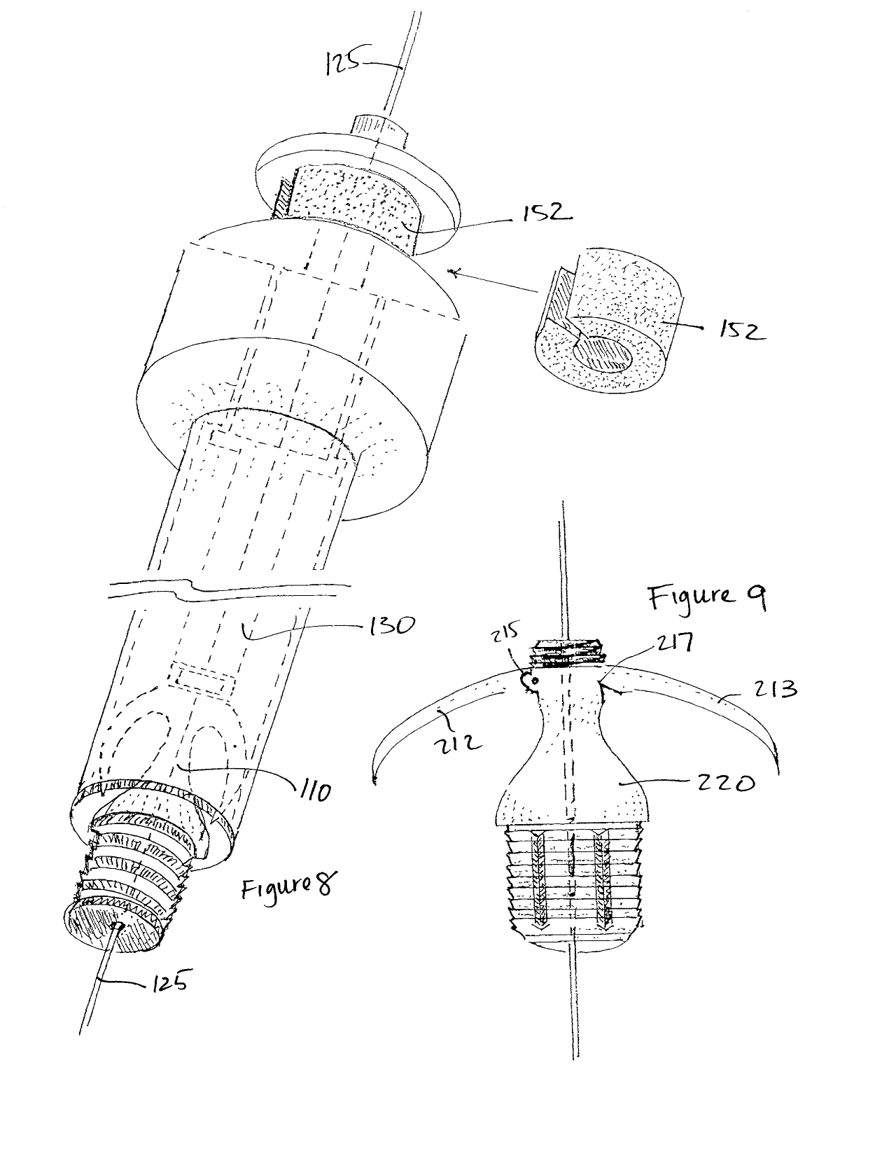

FIG. 8 represents a three-dimensional representation of FIG. 6, above. Un-deployed tissue anchor device (110) is engaged within compression sleeve (140). Guide wire (125) traverses the cannulation channel of the soft tissue anchor device (110) and pushing rod (130). The proximal migration block (152) is shown in both the engaged and disengaged position.

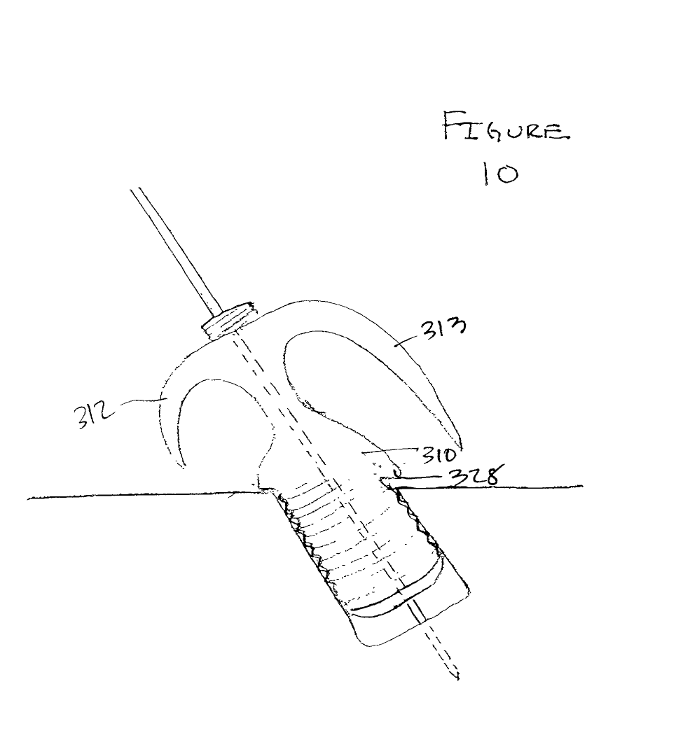

FIG. 9 illustrates a two-dimensional view of an embodiment soft tissue anchor device (220) in accordance with the present disclosure. The device includes two deployed tissue anchoring tongs (212, 213). Each tong includes a bending area or “axilla” that permits the tong to bend to an un-deployed state upon compression and to release to a deployed state. Axilla (215) comprises a pin and arm configuration, such as may be utilized for attaching the earpiece to a common pair of eyeglasses. Axilla (217) comprises a notch-type bending mechanism that facilitates compression of the tongs. One of skill in the art will appreciate that the deployment of the compressed tissue anchoring tongs can occur through various mechanical designs.

Further, in an alternate embodiment, the tissue anchor tongs are asymmetrical. For example, in FIG. 10, tissue anchor device (310) includes tissue-anchoring tongs (312, 313) that have been manufactured for an angled approach to the bone surface. One tong (313) has been lengthened on one side and one tong (312) has been shortened. Blocking ridge (328) is also angled so as to contact the surface of the bone equally on all sides of the anchor device.

A soft tissue anchor device in accordance with the present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed and desired to be secured by United States Letters Patent is:

1. A soft tissue anchor device comprising:
at least one deployable tong for securing soft tissue to bone;
a ribbed anchor portion distal to said at least one deployable tong for securing the soft tissue anchor device into a bone tunnel; and
a blocking ridge distal to said at least one deployable tong.

2. A device in accordance with claim 1, wherein said ribbed anchor portion further comprises at least one retrograde rib and at least one longitudinal rib, said retrograde and longitudinal ribs preventing reverse and rotational movement of the soft tissue anchor device after said ribbed anchor portion is seated within the bone tunnel.

3. A device in accordance with claim 1, further comprising a pushing rod for driving the soft tissue anchor into a bone tunnel.

4. A device in accordance with claim 1, further comprising a compression sleeve.
